(19) United States
(12) Patent Application Publication
(10) Pub. No.: US 2018/0093091 A1
(43) Pub. Date: Apr. 5, 2018

(54) DEVICE FOR FUNCTIONAL ELECTRICAL STIMULATION AND MEASUREMENT OF ELECTROMYOGRAM, COMPRISING MEANS FOR SHORT-CIRCUITING AND EARTHING A PAIR OF ELECTRODES, AND ASSOCIATED TRANSCUTANEOUS ELECTRODE

(71) Applicant: RB PATENTS SARL, Villeneuve (CH)

(72) Inventor: Roland Brodard, Villeneuve (CH)

(21) Appl. No.: 15/554,057

(22) PCT Filed: Feb. 19, 2016
(86) PCT No.: PCT/IB2016/050896

§ 371 Date: Aug. 28, 2017

(30) Foreign Application Priority Data
Feb. 26, 2015 (CH) 00262/15
Feb. 26, 2015 (CH) 00263/15

(51) Int. Cl. A61N 1/04; A61B 5/0488; A61N 1/36

(52) U.S. Cl. CPC A61N 1/0452; A61B 5/04004; A61N 1/36003; A61B 5/0488

(57) ABSTRACT

The present invention relates to a device for functional electrical stimulation and for measurement of electromyogram. The device includes at least one pair of active electrodes intended to be placed on the skin of a user, at least one stimulation module able to generate electric pulses, at least one measurement module able to receive electric pulses, a monitoring and processing unit linked electrically to the stimulation and measurement modules, with the monitoring and processing unit being able to monitor the electric pulses generated by the stimulation module and to process the electric pulses received by the measurement module, and a switching station linked electrically to the stimulation and measurement modules, to the monitoring and processing module, and to the pair of active electrodes. The switching station is able to electrically connect the pair of active electrodes with either the stimulation module, in the case where the pair of active electrodes has to be used to stimulate a muscle of the user, or the measurement module, in the case where the pair of active electrodes has to be used to measure the reaction of the muscle. The switching station is able to momentarily short-circuit and earth ground the pair of active electrodes so as to eliminate any residual voltage at the level of the active electrodes.

### FIELD OF THE INVENTION

The field of the invention is that of functional electrical stimulation (FES) for training the muscle motor function and joint mobility of the upper and lower limbs, in particular for rehabilitation following motor or neuromotor disability, such as for example paraplegia, tetraplegia or hemiplegia.

### PRIOR ART

Neuromuscular electrical stimulation (NMES) is a well-known technique that uses electric currents to activate the nerve endings innervating a muscle and cause the contraction thereof. It is commonly used to enable the contraction of muscles paralyzed following injury to the central nervous system, such as to the medulla, which may cause paraplegia, or to the cerebrum (cerebrovascular accident (CVA), or stroke), which may cause hemiplegia or result in other neuromotor difficulties. It is also used in the field of sports for training muscles and the recovery thereof after stress.

A fundamental distinction should be made between two types of neuromuscular electrical stimulation:

The first, most commonly used type of stimulation, referred to as “conventional” stimulation, in which the various programmed stimulation parameters are purely and simply imposed on a muscle with absolutely no feedback from the muscle activity thus caused. This results in an isometric contraction of the muscle, which contracts but does not shorten, consequently producing no joint movement.

The majority of the stimulators in question offer only a few output channels, usually from two to four channels, in other words from two to four pairs of electrodes.

Among the most advanced of the stimulators, mention may be made of the COMPEX® (U.S. Pat. No. 4,919,139) and the STIWELL® (U.S. Pat. No. 5,285,781) stimulators.

The second type of stimulation, functional electrical stimulation (FES), is specifically designed to produce dynamic muscle contractions, which generate limb joint movements. It should be noted here that the term “functional” is frequently misused, since it is most often incorrectly applied to “conventional” stimulation, such as defined above, that has been enhanced somewhat using “all-or-nothing” remote control contacts.

Indeed, the first devices marketed as FES devices were designed to prevent forefoot drop by stimulating the external popliteal sciatic nerve during walking, particularly in the case of hemiplegia. In this case, a switch, located at the end of the heel of the contralateral shoe, would activate a stimulator carried by the user.

In fact, the term FES should be reserved for multichannel electrical stimulation with real-time closed-loop feedback, designed to generate and to monitor all of the physiological joint movements of the limbs. By way of example, a device of this type designed to train the lower limbs has been produced (patent EP1387712B1 and U.S. Pat. No. 7,381,192B2).

Electromyography (EMG) is a well-known medical technique that makes it possible to record the electrical potentials emitted by a muscle during the voluntary contraction thereof. This can be achieved using two types of EMG, namely surface EMG and invasive EMG.

Surface EMG provides access to all muscles that are relatively close to the surface. During a small contraction, a few potentials from motor units pulsing at low frequency are observed. During a larger contraction, a temporal and spatial recruitment phenomenon is observed. This corresponds to the activation of a larger number of motor units.

The greater the stress, the more these motor units pulse at a higher frequency. Finally, the amplitude of the EMG is proportional to the force of contraction delivered by the muscle.

The use of EMG to control electrical muscle stimulation is well known and has been employed for approximately 40 years (Hansen, G.v.O.: EMG-controlled functional electrical stimulation of the paretic hand, in: Scand. J. Rehab. Med. 11: 189-193, 1979). At the same time, a device offering EMG-controlled neuromuscular stimulation was marketed under the name Automove AM 706, the current version of which is the Automove AM 800. Another, similar device is currently marketed under the name Stiwell med4.

All of the known devices using EMG to control electrical stimulation of a given muscle require five different and specific electrodes to be placed on said muscle, two of said electrodes for electrical stimulation and three electrodes for EMG, i.e. a combination of at least three electrodes, namely two active electrodes for stimulation and for EMG measurement and one grounded reference electrode.

The electrodes used in this type of device must deliver a uniform distribution of electricity over a person's skin below the entire surface of the electrode, that is to say a constant current density per unit area of the electrode, in order to ensure correct coupling. Due to the natural curves of the human body, the electrodes must obviously be flexible not only to adhere perfectly to the contours of the skin under the electrode, but also to accommodate the relative movements of the person's skin.

It is well known that insufficient flexing and conformation of the electrode to the contours of a person may lead to the skin of the person becoming irritated. Electrical “hotspots” due to uneven contact between the electrode and the skin may lead to a cutaneous eruption or a burning sensation. A burning sensation may already be felt by a person a few minutes after application of the electrical signals during the stimulation of nerves and/or muscles, while a cutaneous eruption generally occurs after a longer period of application.

If the entire surface of the electrode is electrically conductive, the entire length of the edge of the electrode may be subject to an electrical “crest effect”, which may lead to a sensation of the edge digging in or a tingling sensation. This effect is avoided if not all of the edge of the border of the electrode is conductive, and is instead insulating.

The majority of flexible transcutaneous electrodes are combined with a flexible and electrically conductive adhesive that allows perfect adhesion of the electrode to the skin of the patient. This adhesive is generally a highly conductive hydrogel.

When such an electrode is used for neuromuscular stimulation, the optimal signal is delivered by a current source in the form of rectangular pulses of two-phase constant current.

Said current can be continuously adjusted from 0 to 100 mA across a load of 2200 ohms. This generally accepted load determines the maximum output voltage of the stimulator, which in this instance is 220 V.

When such an electrode is used to record biological signals, and in particular electromyograms, high electrode conductivity and lower electrode impedance assume particular importance. Indeed, it is well known that in order to carry out an electromyogram measurement under optimum conditions, the circuit of a pair of measurement electrodes placed on the skin ideally requires an impedance within a range of 1-5 kΩ, but at most within a range of 5-10 kΩ, beyond which the quality of the measurement is negatively affected.

In addition, the EMG signal collected at the skin extends from a few microvolts to 2-3 millivolts, exceptionally to 5 millivolts in the case of athletes.

The conditions of use of the electrodes are consequently very different depending on the use to which they are put and when one and the same electrode is used both for nerve and/or muscle stimulation and for recording biological signals, in particular electromyograms, the mechanical and electrical characteristics of the electrodes assume central importance.

It is therefore apparent that the mechanical and electrical characteristics of the electrodes used constitute an element of fundamental and intrinsic importance to a system for stimulating nerves and/or muscles, as well as to a system for measuring biological signals, in particular electromyograms.

The technical features of commercially available electrodes are protected by a large number of patents. Among all of the patents granted, the following may be cited by way of example: U.S. Pat. No. 5,038,796, entitled ELECTRICAL STIMULATION ELECTRODE WITH IMPEDANCE COMPENSATION; U.S. Pat. No. 5,904,712, entitled CURRENT CONTROLLING ELECTRODE; U.S. Pat. No. 4,736,752, which relates to controlling the current density across an electrode by means of defined zones using a conductive ink; U.S. Pat. No. 7,695,430, entitled REVERSE CURRENT CONTROLLING ELECTRODE WITH OVERSIZE BACKING.

Usually, self-adhesive transcutaneous surface electrodes are electrodes designed for single use, or repeated use from one treatment session and/or measurement to the next. However, the service life of these electrodes is limited by a gradual deterioration in their mechanical characteristics, for example in their adhesiveness, and above all in their electrical characteristics due to a decrease in their conductivity and an increase in their impedance. Thus, after a certain number of applications, the electrodes no longer meet the mechanical and electrical needs demanded by their application within a given system. They are then no longer usable and must be thrown away.

These self-adhesive medical electrodes must also, for hygiene reasons in particular, be reserved for application to a single patient.

Finally, due to the fundamental importance of the mechanical and electrical characteristics of the electrodes and the importance of maintaining these characteristics throughout the service life of the electrodes, it would prove to be of great and genuine use to be able to identify and to authenticate, unambiguously, the most suitable electrode selected for a given application and to be able to record, within the electrode itself, data relating thereto that may be processed online using the device to which the electrode is electrically linked.

One solution to this problem has been proposed in US patent application 2014/0235991 A1 or in U.S. Pat. No. 6,146,335 A. It consists in integrating a chip containing authentication data within an electrode system comprising a pair of stimulation electrodes and a pair of measurement electrodes. However, this solution does not allow the electrodes to be authenticated individually, independently of the other electrodes. This solution therefore dictates that the electrodes be applied to the body of the patient in groups and in a predetermined, non-modifiable arrangement.

A first objective of the present invention is therefore to provide a transcutaneous surface electrode that can be used in a device for functional electrical stimulation and electromyogram measurement, that can be identified individually and in which authentication data can be recorded.

Furthermore, due to the use of numerous different electrodes in current devices for functional electrical stimulation and electromyogram measurement, managing all of the stimulation and measurement operations has become particularly complex.

In order to facilitate the management thereof, the use of a single electrode both to stimulate the muscle and to measure the reaction of the muscle to the stimulation has been envisaged in the prior art. Thus, in patent application EP1095670 A1, a neuromuscular electrical stimulator is described in which a stimulation electrode incorporates a sensor, such as an accelerometer or a microphone, in order to measure the muscle reactions caused by the electric pulses generated by the electrode and electronic means for receiving and analyzing the sensor measurements. However, this solution has the disadvantage of being relatively complex to implement. Furthermore, since the sensor does not make direct contact with the muscle, the measurements made by this sensor may not be sufficiently accurate to allow the reactions of the muscle to be analyzed properly. Another known stimulation device, described in patent application WO 02/13673 A2, proposes the use of a single pair of electrodes for both sending electric pulses to the muscle and measuring the voltage originating from the muscle, each electrode potentially being switched to one or the other of the functions by means of a switch. However, this device does not allow precise measurements of the voltage generated by the muscle to be made due to the presence of a residual voltage at the level of the electrodes after a sequence of electric pulses has been sent. This residual voltage, which may reach up to approximately 10 volts, in fact severely disrupts the subsequent measurement made by the electrode, which is of the order of a few millivolts.

A second objective of the invention is therefore to propose a device for functional electrical stimulation and electromyogram measurement using one and the same pair of transcutaneous surface electrodes for carrying out stimulation and measurement and allowing the aforementioned problems to be solved.

### DISCLOSURE OF THE INVENTION

To this end, in accordance with the invention, a device for functional electrical stimulation and electromyogram measurement is proposed, comprising:
at least one pair of active electrodes intended to be placed on the skin of a user;
at least one stimulation module capable of generating electric pulses;
at least one measurement module capable of receiving electric pulses;
a control and processing unit electrically linked to said stimulation and measurement modules, said control and processing unit being capable of controlling the electric pulses generated by said stimulation module and of processing the electric pulses received by said measurement module;
a switching station electrically linked to said stimulation and measurement modules, to said control and processing unit and to said pair of active electrodes, said switching station being capable of electrically connecting said pair of active electrodes with either the stimulation module, in the case where the pair of active electrodes is used to stimulate a muscle of the user, or the measurement module, in the case where the pair of active electrodes is used to measure the reaction of the muscle, the switching operations carried out by said switching station being controlled by said control and processing unit, characterized by the fact that said switching station is capable of momentarily short-circuiting and grounding said pair of active electrodes so as to remove any residual voltage at the level of said active electrodes.

The invention also relates to a transcutaneous surface electrode that can be used in a device for functional electrical stimulation and electromyogram measurement, characterized by the fact that it incorporates an electronic microchip, said microchip containing identification and authentication data relating to the electrode.

Other advantageous configurations of the present invention are defined in dependent claims 2 to 16 and 18 to 20.

### BRIEF DESCRIPTION OF THE DRAWINGS

Other advantages and features of the present invention will be better understood on perusal of a particular embodiment of the invention and with reference to the drawings, in which:

FIG. 1 shows a block diagram of a device in accordance with the present invention;

FIG. 2 is a view similar to FIG. 1, in which the functional components of the switching station used in the device have been shown in detail;

FIG. 3 is a cross section of a transcutaneous surface electrode according to the present invention;

FIG. 4 is a view from below the electrode shown in FIG. 3.

### DETAILED DESCRIPTION

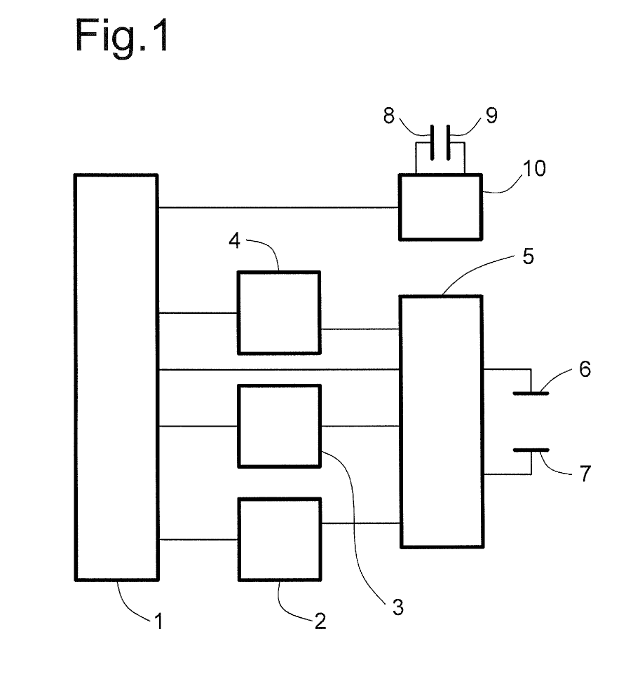

With reference to FIG. 1, which shows, by way of exemplary embodiment, the block diagram of a device according to the present invention described below, a microcomputer 1 is the central programming, data processing and control unit of the overall multichannel system. This microcomputer is connected with various modules or units described below, by means, for example, of an RS232 or RS485 serial link, each module and unit being recognized and identified by its specific address.

The microcomputer 1 is connected with at least one electrical neuromuscular stimulation module 2. This neuromuscular stimulation module, controlled by the microcomputer, contains at least one current source, the output channel of which is floating, i.e. said channel is galvanically isolated from all other electrical or electronic circuits, as well as from ground.

This galvanic isolation (floating output) is also essential between the various output channels of the multichannel system in order to prevent any intracorporeal electrical interaction between the channels when active.

Each stimulation module 2 delivers pulses of two-phase constant current having a duration that can be programmed from 50 to 500 μs. The programmable output current can be continuously adjusted from 0 to 100 mA across a load of 2200 ohms, which load is generally accepted for neuromuscular stimulation, thereby defining a maximum output voltage of 220 V.

Each output channel of a stimulation module 2 is connected to a switching station 5, described further on, which is responsible for managing a pair of electrodes 6 and 7.

The microcomputer 1 is also connected with at least one EMG measurement module 3, the measurement input channel of which is connected to the switching station 5. Each EMG measurement module contains at least one differential operational amplifier. Specifically, EMG measurement between a pair of electrodes extends only from a few microvolts to 2-3 millivolts, exceptionally to 5 millivolts in the case of athletes. As such, this initial signal must be amplified by an amplification factor of the order of 1000 before it can be handed over to an EMG signal processing system, in the present case the microcomputer 1.

The microcomputer 1 is also connected with at least one switching station 5 that manages at least one pair of electrodes 6 and 7. The detailed operation of this switching station will be described further on.

The microcomputer 1 is also connected with at least one unit 4 for managing and controlling the electronic identification and authentication microchips that are incorporated within the electrodes 6 and 7. Said unit, which contains means for managing and controlling said electronic microchips by means of one-wire encrypted data transmission, is connected to the switching station 5. The detailed operation of this device will be described further on.

The microcomputer 1 is additionally connected with a unit for managing and controlling a pair of reference electrodes 8 and 9 of the EMG system, which are connected to the ground of this system. The detailed operation of said unit 10 will be described further on.

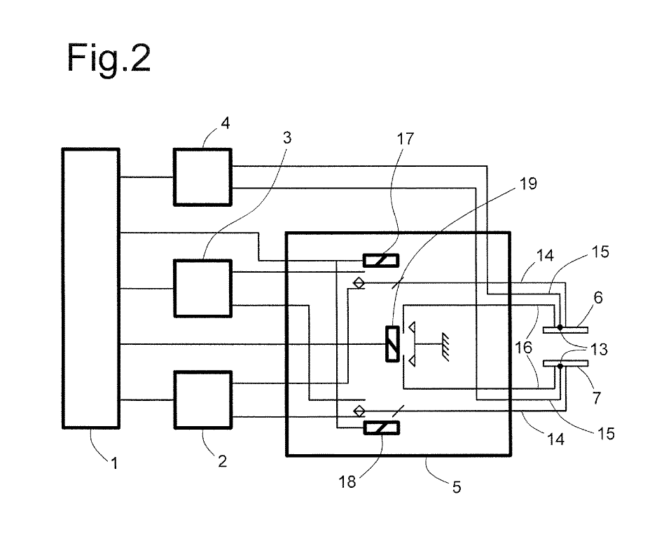

With reference to FIG. 2, which shows, by way of exemplary embodiment, the functional block diagram of the switching station 5 according to the present invention, the switching station 5 contains switching means 17 and 18 and at least one pair of electrodes 6 and 7. Said switching means may advantageously be reed relays, i.e. relays with flexible blades, the contacts of which are enclosed within a glass capsule generally containing dinitrogen. The advantages of this type of relay are a high degree of reliability and a long service life of the order of 10 million open/close cycles, combined with very low contact resistance that is negligible when the contacts are in the closed position and the absence of any leakage current when the contacts are in the open position. Of course, any other suitable mechanical and/or electrical and/or electronic switching means may be used without departing from the scope of the present invention.

In the rest state, the switching means 17 and 18 connect the electrically conductive wires 14 of the pair of electrodes 6 and 7 with the electrical neuromuscular stimulation module 2, thereby allowing the electrostimulation of the muscle positioned below the pair of electrodes 6 and 7.

When the same pair of electrodes 6 and 7 has to be used to carry out an electromyogram measurement on the same muscle, the electrical neuromuscular stimulation module 2 ceases all activity.

Next, in a first instance, by means of the switching element 19 and grounding wires 16, the electrodes 6 and 7 are simultaneously short-circuited and connected to the ground of the device, i.e. to a reference potential, the value of which is generally 0 volts. This action makes it possible to avoid the major drawback of a residual voltage, which may reach a value of up to approximately 10 volts, remaining at the level of the electrodes placed on the skin after a sequence of stimulation pulses of two-phase constant current. Since the voltage originating from the same muscle measured for the purpose of producing the electromyogram thereof extends only from a few microvolts to a few millivolts, it is necessary to remove this residual voltage beforehand by way of the action described above of short-circuiting and grounding the pair of electrodes in question.

In order to remove this residual voltage, the metal sheaths for shielding the cables of the electrodes 6 and 7, surrounding the electrically conductive wires 14, are connected to the ground of the electrical circuitry of the switching station 5 by means of the switching element 19, which may be a reed relay. These shielding sheaths 16 are formed from a plurality of metal wires, constituting an equal number of grounding wires for the device of the invention.

Said momentary action of short-circuiting and grounding the pair of electrodes in question having been duly carried out, it is interrupted by the return of the switching element 19 to the rest position and the switching means 17 and 18 are activated, consequently switching the electrically conductive wires 14 of the electrodes 6 and 7 in order to connect them with the EMG measurement module 3, thus allowing the electromyogram of the muscle to be measured and recorded by means of the same pair of electrodes 6 and 7 left in the same position.

The switching station 5 connects the one-wire electrically conductive wires 15 of the microchips 13 of the electrodes 6 and 7 with the unit 4 for managing and controlling said microchips. The system comprising the management and control unit 4 and the microchips 13 constitutes a master-slave system, where the master is the unit 4 and the slave is the microchip 13.

In this system, the identification and authentication microchip 13 contains, in particular, a read-only memory element that can be wiped electrically, or by any other means, and can be programmed by the user making it possible to store, in a non-volatile manner, application data and additional memory protection means that hold a protected read secret and adjustments to the parameters of the memory by the user.

This master-slave system incorporates security solutions that protect sensitive data under multiple layers of advanced physical security in order to provide the most secure data storage key possible. The master unit 4 contains an SHA-256 coprocessor incorporating a one-wire master function that provides the SHA-256 functionality and the memory required by such a host system for encrypted communication with an SHA-256 one-wire slave, such as for example the microchip 13, and for making use of the latter.

With reference once again to FIG. 1, a unit 10 for managing and controlling a pair of reference electrodes 8 and 9 of the EMG measurement system, connected to the ground of this system, is shown. It is a requirement of reliable and accurate electromyogram measurement that the electronic circuit for electromyogram measurement be connected to ground.

In a multichannel system, it is not necessary for each measurement channel to be connected to ground. It is enough that one neutral reference electrode per person is grounded on a surface of the body that is not electrically involved but also not too remote from the first EMG measurement site. A dorsal position, below the kidneys for example, may be a suitable surface.

To this end, the use of a pair of reference electrodes may prove advantageous, making it easier to measure the electrical impedance of the electrode circuit.

This measurement of the electrical impedance of a pair of electrodes is of great importance. Usually, self-adhesive transcutaneous surface electrodes are electrodes designed for repeated use from one treatment session to the next. However, the service life of these electrodes is limited by a gradual deterioration in their mechanical characteristics, for example in their adhesiveness, and above all in their electrical characteristics due to a decrease in their conductivity and an increase in their impedance. Thus, after a certain number of applications, the electrodes no longer meet the mechanical and electrical needs demanded by their application within a given system. They are then no longer usable and must be thrown away.

It is therefore of great interest to be able to measure, in particular, the impedance of the electrical circuit of a pair of electrodes placed on the skin above a muscle in order to ensure that the measured impedance remains within the usage norm of the system in question.

To this end, the unit 10 for managing and controlling the pair of reference electrodes 8 and 9 of the EMG measurement system contains a current source that delivers a constant-current test signal to the pair of electrodes placed on the skin. When this current is applied to the pair of electrodes it induces a voltage, in accordance with the physical properties of the biological tissue/electrode interface and of the biological tissue through which the current flows between the electrodes. This voltage may be measured and the impedance value may thus be measured in accordance with Ohm's law: Z=V/I.

The same applies for all pairs of active electrodes 6 and 7. Each electrical neuromuscular stimulation module 2 containing a current source that delivers pulses of two-phase constant current having a duration that can be programmed from 50 to 500 μs intended for neuromuscular stimulation may also deliver a constant-current test signal to the pair of electrodes placed on a given muscle and thereby allow the impedance of said pair of electrodes 6 and 7 to be measured in an identical manner to the measurement described for the pair of electrodes 8 and 9.

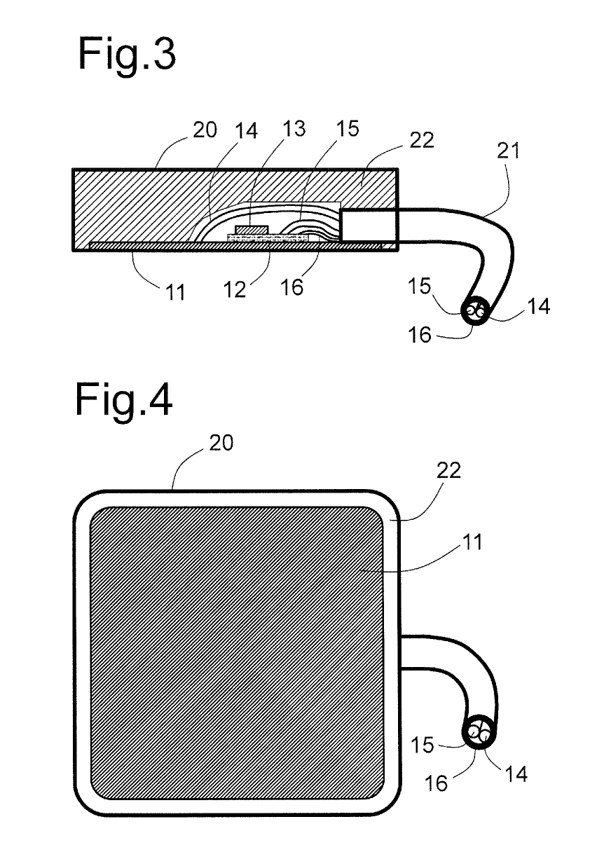

With reference to FIG. 3, which shows, by way of exemplary embodiment, a cross section of a transcutaneous surface electrode 20 incorporating an electronic microchip 13, said electrode is generally composed of at least one electrically conductive flexible element 11 for uniformly distributing the current over its entire surface and the lower face of which is generally coated with a conductive self-adhesive hydrogel.

One end of an electrically conductive wire 14, contained within an electrode cable 21, is brought into contact with the upper face of the flexible element 11.

A flexible printed circuit element 12 is placed with its insulated face on the conductive flexible element 11, while its printed upper face is provided with two separate contact surfaces. One of these surfaces is brought into contact with one end of the electrically conductive wire 15, contained within the electrode cable 21, while the second surface is brought into contact with the end of the shielding sheath 16 of the electrode cable 21.

A one-wire electronic microchip 13 with grounding is placed on the flexible printed circuit element 12, such that its active contact makes contact with the first surface connected to the electrically conductive wire 15 and its grounding contact makes contact with the second surface connected to the shielding sheath 16 of the electrode cable 21.

A non-conductive, insulating flexible element 22 completely covers the upper face of the conductive flexible element 11, to which it may be attached using any suitable adhesive. This non-conductive flexible element also covers and tightly encapsulates the microchip 13 and its connection elements 12 as well as the end of the electrically conductive wire 14 that makes contact with the conductive flexible element 11 and an insertion of the electrode cable 21.

This non-conductive, insulating flexible element 22 also prevents any unwanted contact with the conductive element 11 and the connection elements of the electrode and of the microchip.

The electrode cable 21 contains two electrically conductive wires, the wire 14 linked to the conductive flexible element 11 of the electrode and the wire 15 linked to the microchip 13 via the element 12, this cable also contains a flexible metal shielding sheath 16. This shielding sheath is essential when the electrode is used for electromyogram measurement and it is also used for grounding the microchip 13 via the element 12.

Indeed, when the electrode cable is connected to the switching station 5, the shielding sheath 16 is connected to the common ground of the overall device.

With reference to FIG. 4, which shows the lower face of the electrode 20 that is intended to be applied to the skin of a person, with its electrically conductive flexible element 11 for uniformly distributing the current over its entire surface and the lower face of which is generally coated with a conductive self-adhesive hydrogel. Usually, the non-conductive, insulating flexible element 22 that completely covers the upper face of the conductive flexible element 11 also continues over the entire perimeter of the electrode, thus creating an insulated peripheral zone that prevents an electrical “crest effect” along the edge of the electrically conductive element 11.

By way of exemplary embodiment of the invention, the microchip used may be the DS28E25 DeepCover Secure Authenticator with 1-Wire SHA-256 and 4 kB user EEPROM by Maxim Integrated Products, Inc.

This microchip, the dimensions of which are of the order of 6 mm×6 mm×0.9 mm thick, may be easily incorporated within the electrode without modifying its flexibility or its operational capability. It also has the advantage of offering a one-wire solution, a single electrically conductive wire being used both to supply power to the chip at 3.3 V and for the communication of data between the chip and the host system.

The DS28E25 chip incorporates security solutions that protect sensitive data under multiple layers of advanced physical security in order to provide the most secure data storage key possible. This DS28E25 chip combines heavily encrypted bidirectional secure “challenge-response” functionality using means based on the FIPS 180-3 specified“Secure Hash Algorithm (SHA-256)”.

The DS28E25 chip in particular contains a 4 kB EEPROM read-only memory element that can be wiped electrically and can be programmed by the user making it possible to store, in a non-volatile manner, application data and additional memory protection means that hold a protected read secret for SHA-256 operations and adjustments to the parameters of the memory by the user.

Each DS28E25 chip has its own guaranteed unique 64-bit ROM identification number (ROM ID) that is factory programmed into the chip. This unique ROM ID is used as an essential input parameter for encryption operations and is also used as an electronic serial number for a given application.

A bidirectional security model allows two-way authentication between a host system and the integrated slave DS28E25. The authentication of the slave DS28E25 in the direction of the host is used by the host system to validate, in complete security, that an attached or integrated DS28E25 chip is authentic.

The authentication of the host system in the direction of the slave DS28E25 is used to protect the user memory of the DS28E25 chip from being modified by an inauthentic host. The SHA-256 message authentication code (MAC), which the DS28E25 chip generates, is computed from data in the user memory, namely a secret on the chip, a host controller random challenge, and the 64-bit ROM ID.

The DS28E25 chip communicates via a one-wire bus at overdrive speed. Communication takes place according to the one-wire protocol, with the ROM ID acting as a node address in the case of a network of multiple DS28E25 one-wire chips.

The DS2465 element incorporates security solutions that protect sensitive data under multiple layers of advanced physical security in order to provide the most secure data storage key possible. This DS2465 element is an SHA-256 coprocessor incorporating a one-wire master function that provides the SHA-256 functionality and the memory required by such a host system for communication with an SHA-256 one-wire slave, such as for example the DS28E25 element, and for making use of the latter. In addition, the DS2465 element performs protocol conversion between the I²C master and each of the connected SHA-256 one-wire slaves.

In order to control a one-wire line, user-adjustable internal timers allow the processor of the host system to avoid having to generate time-critical one-wire waveforms, supporting both standard and overdrive one-wire communication speeds. A one-wire line may be powered down by control software.

Strong features allow one-wire power delivery to one-wire devices such as EEPROMs. When the DS2465 element is not in use, it may be placed in sleep mode, in which its power consumption is minimal.

A description of the usage procedures of the overall system is given below by way of example. All of the procedures are controlled by the microcomputer 1, as the central programming, data processing and process control unit of the overall multichannel system.

At the start of a treatment session and/or EMG measurement, while the system is in neuromuscular stimulation configuration with the stimulation modules 2 connected to their respective electrodes 6 and 7 by means of the switching station 5, the electrical impedance of each of the pairs of active electrodes 6 and 7 is measured and recorded, including by means of the unit 10 of the pair of reference electrodes 8 and 9.

Once this operation has been achieved and acknowledged, it is possible either to commence directly with the functional electrical stimulation (FES) of the muscles or to carry out an EMG measurement of said muscles beforehand. In the latter case, an operation of short-circuiting and grounding all of the pairs of active electrodes 6 and 7 must always be carried out in neuromuscular stimulation configuration, but with the latter then having been deactivated, prior to any EMG measurement, by means of the switching station 5 in order to remove any residual voltage at the level of the electrodes 6 and 7. Once this operation has been accomplished and acknowledged, it is possible, by means of the switching station 5, to disconnect each pair of electrodes 6 and 7 from the stimulation modules 2 in order to switch and connect them to the corresponding EMG measurement modules 3. Said measurement is then carried out.

For example, an EMG measurement may be made prior to an FES session, and a new EMG measurement may be made following said FES session.

Following the description that has just been given with a view to illustrating the manner in which the invention may advantageously be put into practice, it should be noted that the invention is not limited to this embodiment.

Multiple variant embodiments of an FES device combined with the corresponding electromyogram measurement by means of one and the same single pair of electrodes and a single placement of said electrodes on a given muscle may be envisaged in the field of those skilled in the art without departing from the scope of the present invention as defined in the appended claims.

Furthermore, multiple other variant embodiments of an electrode incorporating an identification and authentication microchip may be envisaged in the field of those skilled in the art without departing from the scope of the present invention as defined in the appended claims.

1. A device for functional electrical stimulation and electromyogram measurement, comprising:
at least one pair of active electrodes intended to be placed on the skin of a user;
at least one stimulation module capable of generating electric pulses;
at least one measurement module capable of receiving electric pulses;
a control and processing unit electrically linked to said stimulation and measurement modules, said control and processing unit being capable of controlling the electric pulses generated by said stimulation module and of processing the electric pulses received by said measurement module;
a switching station electrically linked to said stimulation and measurement modules, to said control and processing unit and to said pair of active electrodes, said switching station being capable of electrically connecting said pair of active electrodes with either the stimulation module, in the case where the pair of active electrodes is used to stimulate a muscle of the user, or the measurement module, in the case where the pair of active electrodes is used to measure the reaction of the muscle, the switching operations carried out by said switching station being controlled by said control and processing unit,
wherein said switching station is capable of momentarily short-circuiting and grounding said pair of active electrodes so as to remove any residual voltage at the level of said active electrodes.

2. The device as claimed in claim 1, wherein the stimulation module is electrically connected to the switching station by means of a floating output channel such that said channel is galvanically isolated from all other electrical or electronic circuits, as well as from ground.

3. The device as claimed in claim 1, wherein each active electrode is electrically linked to the switching station by means of at least two wires, a conducting wire and a grounding wire, respectively.

4. The device as claimed in claim 3, wherein the switching station comprises first switching means that are capable of connecting the conducting wires of said pair of active electrodes with an output of the stimulation module or with an input of the measurement module.

5. The device as claimed in claim 4, wherein the switching station comprises second switching means that are capable of connecting the grounding wires of said pair of active electrodes with the ground of the device.

6. The device as claimed in claim 4, wherein said first and/or second switching means are reed relays.

7. The device as claimed in claim 3, wherein the grounding wire forms an integral part of a sheath for shielding an electrode cable, said cable comprising the conducting wire and said shielding sheath surrounding said conducting wire.

8. The device as claimed in claim 1, further comprising a first management and control unit, which unit is electrically linked to the control and processing unit, and at least one pair of reference electrodes that are intended to be placed on the skin of the user at a reference location and electrically linked to said first management and control unit, said first management and control unit being capable of delivering a constant current to said pair of reference electrodes and of measuring the impedance value of said pair of reference electrodes.

9. The device as claimed in claim 1, wherein the stimulation module is capable of delivering a constant current to the pair of active electrodes so as to allow the impedance value of said pair of active electrodes to be measured.

10. The device as claimed in claim 1, wherein each active electrode incorporates an electronic identification and authentication microchip that is connected, by means of a one-wire data transmission link, to a unit for managing and controlling said microchips, said management and control unit being capable of managing and of controlling said microchips.

11. The device as claimed in claim 10, wherein the one-wire data transmission link is an electrically conductive wire that also delivers an electric supply current to the microchip.

12. The device as claimed in claim 11, wherein each active electrode comprises at least one electrically conductive flexible element capable of transmitting current uniformly over its entire surface, said conductive element having a lower face, intended to come into contact with the skin of the user and preferably coated with a conductive self-adhesive hydrogel, and an upper face, which is electrically linked to the switching station by means of the conducting wire.

13. The device as claimed in claim 12, wherein each active electrode comprises a flexible printed circuit element, a lower, electrically insulated, face of which is placed in contact with the electrically conductive element, and an upper face of which has at least two separate contact surfaces, a first contact surface, electrically linked to the management and control unit by means of the one-wire link, and a second contact surface, linked to the ground of the device by means of a grounding wire, respectively, and wherein the microchip is placed on the upper face of said printed circuit element such that one of the contacts of said microchip makes contact with said first contact surface and that another contact of said microchip makes contact with said second contact surface.

14. The device as claimed in claim 13, wherein the grounding wire forms an integral part of a sheath for shielding an electrode cable, said cable comprising the one-wire link and said shielding sheath surrounding said one-wire link.

15. The device as claimed in claim 10, wherein each microchip contains a read-only memory element that can be wiped electrically, or by any other means, and can be programmed by the user making it possible to store, in a non-volatile manner, application data and additional memory protection means that hold a protected read secret and adjustments to the parameters of the memory by the user, the bidirectional transmission of the data being encrypted.

16. The device as claimed in claim 10, wherein a system comprising the management and control unit and the microchips constitutes a master-slave system, where the master is the unit and the slave is the microchip.

17. A transcutaneous surface electrode that can be used in a device for functional electrical stimulation and electromyogram measurement, the transcutaneous surface electrode incorporating an electronic microchip, said microchip containing identification and authentication data relating to the electrode.

18. The electrode as claimed in claim 17, further comprising at least one electrically conductive flexible element capable of transmitting current uniformly over its entire surface, said conductive element having a lower face, intended to come into contact with the skin of the user and preferably coated with a conductive self-adhesive hydrogel, and an upper face, to which a flexible printed circuit element is attached, a free face of said printed circuit element, which does not make contact with said upper face, having at least one first contact surface, from which data pass, the microchip being positioned on said free face such that one of the contacts of said microchip makes contact with said first contact surface.

19. The electrode as claimed in claim 17, wherein each microchip contains a read-only memory element that can be wiped electrically, or by any other means, and can be programmed by the user making it possible to store, in a non-volatile manner, application data and additional memory protection means that hold a protected read secret and adjustments to the parameters of the memory by the user, the bidirectional transmission of the data being encrypted.

20. An assembly, comprising the transcutaneous surface electrode as claimed in claim 18, and an electrode cable comprising at least two electrically conductive wires, one of the electrically conductive wires making contact with the upper face of the conductive element of the electrode and the other electrically conductive wire making contact with the free face of the printed circuit element of the electrode, and wherein said cable additionally comprises a shielding sheath surrounding said electrically conductive wires, said shielding sheath being capable of placing the conductive element and the microchip at a reference electrical potential.
